(19) United States
(12) Patent Application Publication
(10) Pub. No.: US 2026/0133174 A1
(43) Pub. Date: May 14, 2026

(54) Pressure Sensing Dissolved Inorganic Carbon

(71) Applicant: Woods Hole Oceanographic Institution, Woods Hole, MA (US)

(72) Inventors: Jason KAPIT, North Falmouth, MA (US); Anna MICHEL, Falmouth, MA (US)

(21) Appl. No.: 19/388,053

(22) Filed: Nov. 13, 2025

Related U.S. Application Data
(60) Provisional application No. 63/719,739, filed on Nov. 13, 2024.

(51) Int. Cl. G01N 33/18

(52) U.S. Cl. CPC G01N 33/18

(57) ABSTRACT

Detecting dissolved inorganic carbon (DIC) in a liquid by directing liquid through a conduit, acidifying a portion of the liquid to a pH at least as low as 4.0 to form an acidified liquid portion, and exposing a first surface of a membrane to the acidified liquid portion, the membrane being permeable to CO₂ gas but impermeable to the liquid. A second surface of the membrane is exposed to a first headspace to collect CO₂ passed through the membrane, and the amount of CO₂ in the headspace is quantified by a pressure sensor to obtain a sensed CO₂ pressure. The amount of DIC in the liquid is determined based on the sensed CO₂ pressure.

### CROSS-REFERENCE TO RELATED APPLICATION

This application claims priority to U.S. Provisional Application No. 63/719,739 filed 13 Nov. 2024. The entire contents of the above-mentioned application are incorporated herein by reference as if set forth herein in entirety.

### FIELD OF THE INVENTION

This invention relates to detecting dissolved inorganic carbon in a liquid such as seawater.

### BACKGROUND OF THE INVENTION

When CO₂ dissolves in water, it proportions into the three species: carbon dioxide gas (CO₂), bicarbonate ions (HCO₃⁻), and carbonate ions (CO₃²⁻), the sum of which equals total DIC (dissolved inorganic carbon). The ratio of these species is controlled by the pH of the water and this dependency is often exploited to measure total DIC. Accordingly, rather than using three separate sensors to measure each species independently, it is possible to measure DIC by first acidifying a water sample below pH 4 to convert all the DIC to CO₂, and then quantifying the total CO₂. See, e.g., U.S. Pat. No. 10,830,692 by Wang et al. for the critical role played by the marine carbon dioxide system and for in situ sensing systems such as the Channelized Optical System (CHANOS), also referred to as Dual-channel Modularized Autonomous System (D-MAS), that is capable of making high-resolution, simultaneous measurements of at least two parameters such as total dissolved inorganic carbon (DIC) and pH in seawater.

There has also been interest in removing CO₂ from seawater. See, e.g., Seoni Kim et al., “Asymmetric chloride-mediated electrochemical process for CO₂ removal from oceanwater”, Energy Environ. Sci. 2025, vol. 16, pp. 2030-2044 (“Kim et al.”).

No commercial DIC sensors currently exist that are affordable for widespread use. Creating a scalable, widely-deployable sensor with high commercial potential would be a major step to achieving DIC sensing of natural waters, and it would constitute a substantial advancement for ocean science and monitoring.

### SUMMARY OF THE INVENTION

An object of the present invention is to provide accurate sensing of dissolved inorganic carbon with lower-cost systems.

Another object of the present invention is to provide such systems which are deployable underwater yet are sufficiently accurate for bench-top applications.

This invention features a system and method which detect dissolved inorganic carbon (DIC) in a liquid by directing liquid through a conduit, acidifying a portion of the liquid to a pH at least as low as 4.0 to form an acidified liquid portion, and exposing a first surface of a membrane to the acidified liquid portion, the membrane being permeable to CO₂ gas but impermeable to the liquid. A second surface of the membrane is exposed to a first headspace to collect CO₂ passed through the membrane, and the amount of CO₂ in the headspace is quantified utilizing a pressure sensor in the headspace to obtain a sensed CO₂ pressure. The amount of DIC in the liquid is determined based on the sensed CO₂ pressure.

In one embodiment, the pressure sensor is a differential pressure sensor positioned between the first headspace and a second headspace which lacks CO₂. In some embodiments, the acidifying is accomplished by electro-acidification and, in other embodiments, by utilizing at least one reagent.

This invention also features a system to detect dissolved inorganic carbon (DIC) in a liquid, including a conduit having a first opening to receive a liquid, a passage, and a second opening to discharge the liquid. An acidification module is configured to interact with the liquid in the passage to acidify a portion of the liquid to a pH at least as low as 4.0 to form an acidified liquid portion. A membrane having a first membrane surface is configured to be exposed to the acidified liquid portion, the membrane being permeable to CO₂ gas but impermeable to the liquid and having a second membrane surface configured to be exposed to a first headspace to collect CO₂ passed through the membrane. A pressure sensor is configured to quantify the amount of CO₂ in the headspace to obtain a sensed CO₂ pressure, and a microprocessor configured to determine the amount of DIC in the liquid based on the sensed CO₂ pressure.

### BRIEF DESCRIPTION OF THE DRAWINGS

To enable a better understanding of the present invention, and to show how the same may be carried into effect, certain embodiments of the invention are explained in more detail with reference to the drawings, by way of example only, in which:

FIG. 1 is a schematic diagram of a DIC sensing system having a pressure sensor according to the present invention;

FIG. 2 is a schematic illustration of a system utilizing a planar membrane;

FIG. 3 is a schematic illustrations of a system utilizing a tubular membrane;

FIG. 4A is a schematic diagram of a tubular membrane in an electrochemical cell and a gas circulation loop according to another embodiment of the present invention;

FIG. 4B is a schematic diagram of a system having parallel streams according to yet another embodiment of the present invention for separate acidification and basification; and

FIG. 5 is a schematic diagram of sequential basification and acidification with a differential pressure sensor according to the present invention.

### DETAILED DESCRIPTION OF THE PRESENTLY PREFERRED EMBODIMENTS

This invention may be accomplished by an in situ dissolved inorganic carbon (DIC) sensing system for water measurement and analysis applications utilizing sample acidification followed by CO₂ detection utilizing a pressure sensor. The sensing system is small, inexpensive, and has low power requirements, thus enabling scalability, as well as operation on platforms in many environments. Its small size will enable it to be scaled onto arrays of ocean platforms such as AUVs (autonomous underwater vehicles) and buoys which could for the first time enable DIC sensing in the ocean at large scales.

A number of embodiments utilize electro-acidification instead of reagents, enabling the DIC sensing system to be reagent-free. Furthermore, its reagent-free nature would eliminate what has historically been a critical barrier to the scalability and ease of use for DIC sensors as well as to autonomous long-term operation.

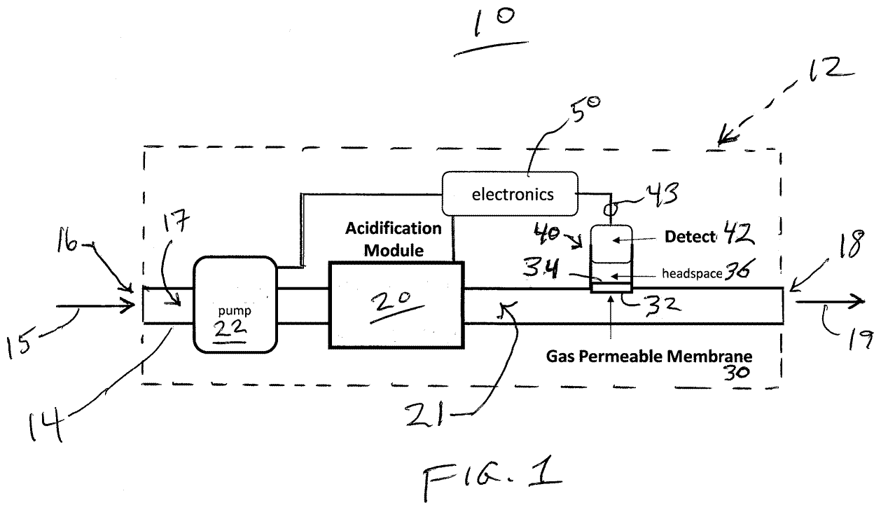

A system 10, FIG. 1, according to the present invention includes a housing 12, shown in dashed lines. A conduit 14 has a first opening 16 to receive a liquid (represented by flow arrow 15), a passage 17, and a second opening 18 to discharge the liquid as indicated by flow arrow 19. An acidification module 20 received liquid via pump 22 and is configured to interact with the liquid in the passage 17 to acidify a portion of the liquid to a pH at least as low as 4.0 to form an acidified liquid portion 21.

A membrane 30 has a first membrane surface 32 that is exposed to the acidified liquid portion 21, the membrane being permeable to CO₂ gas but impermeable to the liquid and having a second membrane surface 34 that is exposed to a first headspace 36 to collect CO₂ passed through the membrane 30. A pressure sensor 42 within a CO₂ detection module 40 quantifies the amount of CO₂ in the headspace 36 as a simple pressure reading to obtain a sensed CO₂ pressure. Electronics module 50 is electrically connected to pump 22, acidification module 20 and detection module 40.

In one construction, electronics module 50 is enclosed in a pressure-resistant, water-tight housing and includes a controller board for the pump motor, a current or voltage source for the acidification module, a power source such as a battery, an AD converter for reading thermistors or other sensors, and a microprocessor for sequencing the whole system, determining DIC from sensed CO₂ pressure as described below, and collecting and storing data. The microprocessor converts sensed CO₂ pressure to dissolved CO₂ concentration using Henry's Law solubility constants adjusted for the temperature, salinity, and pressure of the water. After this conversion, it is assumed that the dissolved CO₂ concentration equals the concentration of DIC.

Overview: in one construction, the pump 22 collects ambient water through opening 16 and pumps the water into the acidification module 20 at a rate of ˜1-10 mL/min. The acidification module 20, which is an electrochemical module in some embodiments, serves to acidify the passing water to pH<4; this acidification step converts all the DIC in the sample water to CO₂ gas. After being acidified, the solution enters the CO₂ detection module 40, where it flows past a gas-permeable (and water-impermeable) polymer membrane 30. On the opposite side of the membrane is a dry headspace 36 containing a pressure sensor 42 as a miniature CO₂ gas detector. Accordingly, as the acidified sample 21 flows past the membrane 30, the CO₂ gas in the headspace 36 equilibrates with CO₂ gas in the water. As this process occurs, the CO₂ content of the headspace is measured by the CO₂ detector, and once full equilibration is reached, the CO₂ detector signal 43 is proportional to the DIC in the original sample. The sensing system operates with continuous flow in some constructions and, in other constructions, by periodic (discontinuous) flow.

Acidification module 20: sensing systems according to the present invention operate by first pumping water through an acidification module. There are two embodiments for this module: (a) traditional liquid acidification utilizing one or more reagents, and (b) electro-acidification. Traditional acidification: In some embodiments, a traditional liquid acid dosing module including a liquid acid pump motor is used for mixing acid with sample seawater to convert all the DIC to CO₂. Liquid reagents are consumables that increase complexity and chances of user error.

Electro-acidification: An electrode-based water acidification approach eliminates the need for a liquid reagent and presents the possibility for a fully solid-state DIC sensor. In one construction, a bismuth-based electrochemical cell which modulates the pH of one or two sample streams, which become acidified or basified.

Specifically, acidification can also be achieved electrochemically through electrolysis or electrodialysis. In electrolysis, current applied to electrodes splits water to release hydrogen. However, this process also produces chlorine gas which aggressively reacts or interferes with other senor components. Electrodialysis mitigates this by using a bipolar membrane to split water catalytically into protons and hydroxyl ions, with each reporting to a different flow stream so that one stream is acidified and the other becomes basic. The limitations of such a process are the need for membranes, anolyte and catholyte solutions, high cell voltages, and, possibly, generation of gases (H₂, O₂ and/or Cl₂).

One electro-acidification approach is described in Kim et al. as cited above in the Background. Applying a voltage to an electrochemical cell composed nominally of, but not limited to, Bi and AgCl electrodes serves to acidify seawater. The voltage can be reversed to basify the seawater and regenerate the electrodes. This electrochemical technique holds great promise for a compact solid-state DIC sensor in that: (1) it is small (˜1 cm) and low power (<100 mW); (2) the reaction is fully reversible meaning the electrodes can be regenerated in seawater; (3) it provides for a plethora of sensor architecture options to explore; and (4) it enables frequent “zero” measurements to eliminate sensor drift.

Membrane 30: After the sample stream is acidified, it passes by a gas permeable/water-impermeable membrane, composed nominally but not limited to Teflon, PDMS, or PTMSP, through which the evolved CO₂ enters a tiny gas headspace. After a period of time, the pCO₂ in the headspace equilibrates with the pCO₂ in the water through Henry's Law. Different embodiments have different membrane form factors and geometries (e.g. flat vs. tubular) to optimize the gas extraction from the sea water as well as overall sensor performance.

Pressure sensor: After the gas equilibrates in the headspace, it is quantified by a new detection approach using a simple pressure sensor. One suitable pressure sensor for use according to the present invention is a barometric pressure and temperature sensor Model No. ICP-10125 available from Invensense, an affiliate of TDK Corporation.

Since the conversion of DIC in standard seawater to free CO₂ theoretically results in a pCO₂ change of ˜0.07 atm, it is a realization of the present invention that an appropriate pressure sensor could theoretically measure that change with high accuracy. The key advantage of a pressure detector over more traditional CO₂ detectors would be its extremely high long-term accuracy, which could be as good as 0.05%. If two pressure sensors (or a single differential sensor) were to monitor the acidified and basified sample streams, the difference between the two readings would theoretically be only the evolved CO₂, resulting in a very robust detection approach.

By comparison, conventional CO₂ detectors include numerous COTS MEMS (commercial off-the-shelf microelectromechanical systems) technologies that are currently marketed for CO₂ gas quantification. These technologies include detectors based on non-dispersive infrared absorption (NDIR) such as the Sensair K33 1CB sensor, photoacoustics (PA) such as the Sensirion SCD41 sensor, and gas thermal conductivity (TC) such as the Sensirion SCD31 sensor.

In its most basic form, a system according to the present invention includes a Teflon AF membrane separating the acidified sample water from a headspace containing a pressure sensor as a CO₂ sensor. In this configuration, Henry's Law ensures that the gas in the headspace is always driven towards equilibrium with the gas in the seawater. For CO₂ this can be written as:

$${p}_{C {O}_{2}} = H {C}_{C {O}_{2}} \qquad Equation 1$$

where C_{CO2} is the free CO₂ concentration in the water, p_{CO2} is the CO₂ partial pressure in the headspace, and H is Henry's constant for CO₂ in seawater. Thus, the partial pressure in the headspace can be used to determine the total DIC concentration, since under acidic conditions C_{CO2}=C_{DIC}.

When there is a step change in the C_{CO2} in the water (such as when the sample is acidified), the response of the pCO₂ gas in the headspace is governed by first-order kinetics, which has a solution of the form:

$${p}_{C O 2} ( t ) = H Δ {C}_{C O 2} + exp ( t / τ ) \qquad Equation 2$$

and the t₆₃ equilibration time, τ, is time given by:

$$τ = \frac{{V}_{h}}{k} \qquad Equation 3$$

Here k is a constant based on the membrane's geometry and its permeability to CO₂, and Vₕ is the volume of the headspace. Thus, the key to optimizing the pressure sensor's response time is maximizing membrane permeability and minimizing the volume of the headspace (i.e. the size of the CO₂ sensor).

It should be noted that the Henry's law constant itself is dependent on the hydrostatic pressure, temperature and salinity of the water. Sensors for one or more of those parameters are included in certain constructions of systems according to the present invention.

Since [CO₂]=[DIC] at low pH, this implies that at a given hydrostatic pressure P, the DIC concentration can be determined from the measured partial pressure and the Henry's law constant at a reference ambient pressure of 1 atm and temperature T:

$$[ DIC ] = \frac{{p}_{{CO}_{2}} ( P )}{{H}_{{P}^{r e f}} exp [ \frac{{υ}_{{CO}_{2}}}{R T} ( P - {P}^{r e f} ) ]} \qquad Equation 4$$

A microprocessor in electronics module 50 or other type of controller is configured to determine the amount of DIC in the liquid based on the sensed CO₂ pressure.

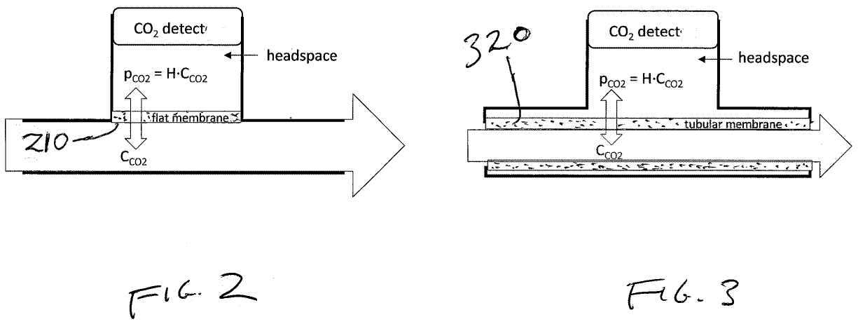

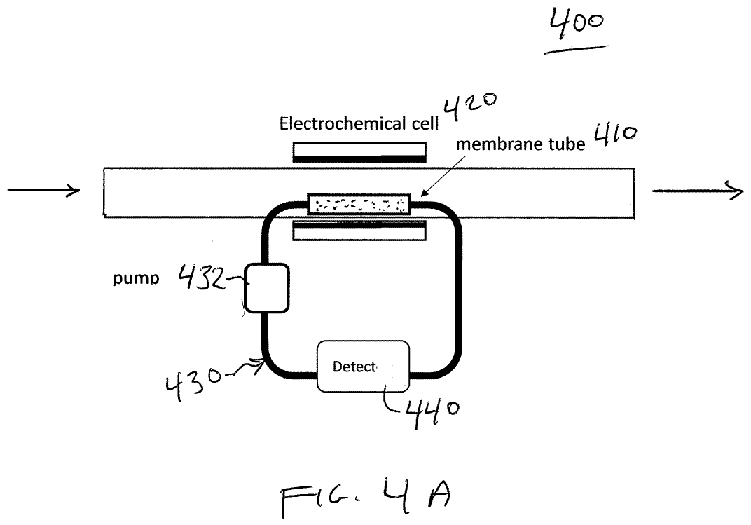

FIGS. 2-3 depict one CO₂ detection approach according to the present invention using different membranes. As acidified seawater containing CO₂ passes a membrane unit 210, FIG. 2, the CO₂ gas equilibrates with the gas in the headspace according to Henry's Law. Membrane unit 210 is planar in some constructions and is curved in other constructions.

Alternatively, a tubular membrane 320, FIG. 3, is utilized for CO₂ extraction. As described by Kapit and Michel, the present inventors, in “Dissolved gas sensing using an anti-resonant hollow core optical fiber”, Applied Optics (2021), Vol. 60, No. 33, pp. 10354-10358, 90% equilibration across a Teflon AF or suitable material tube into a ˜1 mL headspace can be achieved in ˜8 min. Alternative high-permeability membrane materials such as silicone exist, but they have been found to saturate or swell with water and gas when exposed to ocean pressure. In contrast, Teflon AF has a history of functioning successfully in previous ocean applications.

It is noted that many COTS sizes for Teflon AF tubes can withstand hydrostatic pressure of up to 1000 meters, and flat membranes with support structures are utilized at ocean depths of >2000 m. In these configurations, the headspace pressure is always controlled by Henry's law and remains at 1 atm or less despite the high ambient hydrostatic pressure.

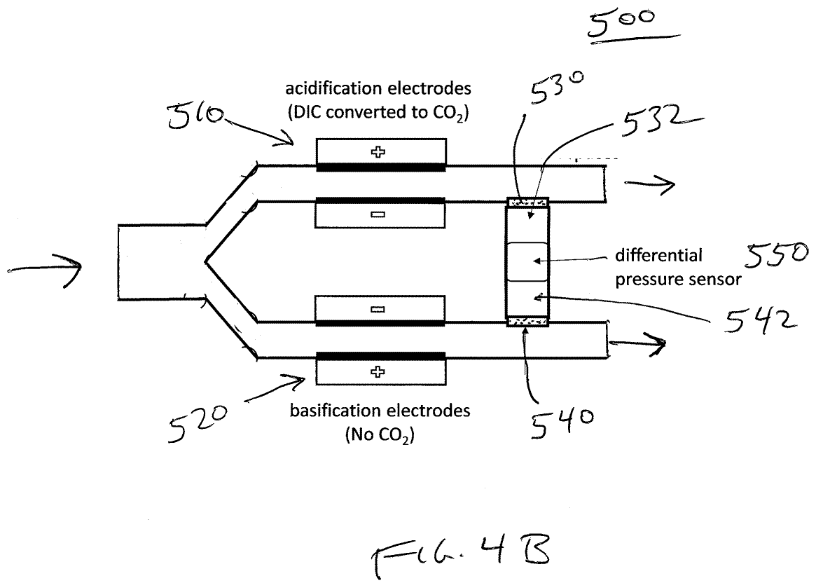

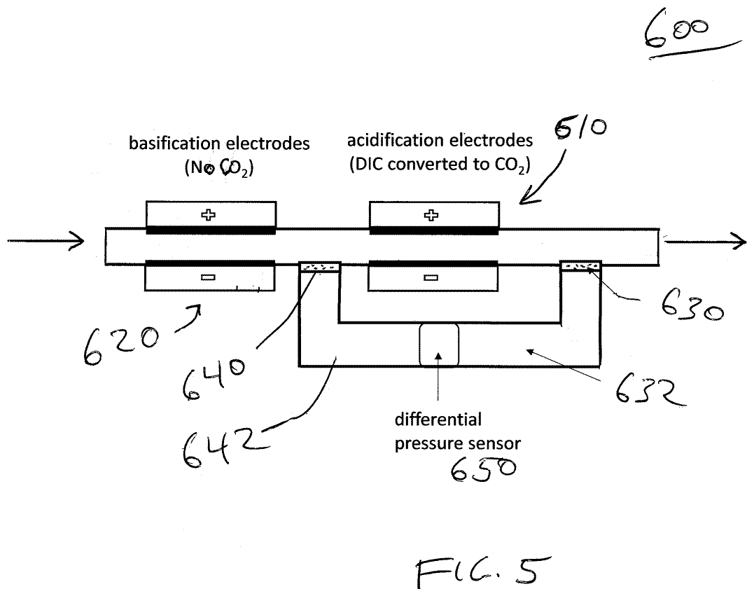

There are several possible alternative sensor architectures including the additional architectures depicted in FIGS. 4A-5. Each architecture presents a likely tradeoff between the sensor's complexity and performance, with higher performance resulting in slightly higher complexity. A tubular membrane 410, FIG. 4A, is disposed inside (or at the effluent region) of the electrochemical cell 420, and a gas circulation loop 430 equilibrates with the CO₂ in the passing water. Detection module 440 includes a pressure sensor according to the present invention.

The architecture of system 400, FIG. 4A, is similar to FIG. 1 with the exception that the sample water flows past the outside of the Teflon AF tube 410 instead of inside, and the gas headspace, which is now on the inside of the tube, is circulated in a loop 430 either clockwise or counter-clockwise by pump 432. This architecture prevents the water from having to flow through a small capillary, and it also presents options for increasing the membrane surface area exposed to the water, as well as achieving faster headspace equilibration.

System 500, FIG. 4B, has acidification and basification occurring on two parallel flow streams on the same sample water, and a differential measurement is made between the extracted pCO₂ on the acidified side having acidification electrodes 510 and membrane 530, and zero pCO₂ on the basified side having basification electrodes 520 and membrane 540. The architecture depicted in FIG. 4B enables differential measurements to be achieved, which could aid mitigating interferences and drifts. Here, a single sample stream is split to flow through two electrochemical cells which are identical except for polarity, one of which acidifies the sample while the other basifies it. Afterwards, the two sides equilibrate two headspaces, a first headspace 532 with pCO₂=C_{DIC}, and the other headspace 542 with pCO₂=0. While the differential detection could be achieved utilizing two identical CO₂ sensors or pressure sensors, it could be very advantageous to use a differential pressure sensor 550 to monitor the pCO₂ change. With this architecture, the only difference on either side of such a pressure sensor 550 would be the pCO₂ in the headspace, which could theoretically result in a very accurate measurement.

Yet another architecture utilizing a single conduit is illustrated for system 600, FIG. 5, having basification electrodes 620 which results in zero CO₂ crossing membrane 640 into second headspace 642, and downstream acidification electrodes 610 which result in all available CO₂ passing through membrane 630 into first headspace 632. A differential pressure sensor 650 operates in a similar manner to sensor 550, FIG. 4B. The order of the basification and acidification electrodes can be equivalently switched, with the acidification electrodes occurring first in the flow path, and the basification electrodes second.

It is noted that each of these architectures can be constructed using any of the pressure sensor types, differential or absolute. Each architecture allows for a zero measurement to be performed before, or alongside, each DIC measurement, thereby mitigating many common drift issues which typically influence other sensors.

In some embodiments, the full sensing system, including, but not limited to, the pump, the electro-acidification module, the CO₂ detection module, and their associated components are optimized for a submersible DIC sensor that is robust to ocean pressure and results in high accuracy and resolution, while minimizing the sensor's response time.

In some embodiments, the full sensing system, including, but not limited to, the pump, the electro-acidification module, the CO₂ detection module, and their associated components are optimized for use as a laboratory benchtop sensor.

In some embodiments, the full sensing system, including, but not limited to, the pump, the electro-acidification module, the CO₂ detection module, and their associated components are optimized for use as a field-portable sensor.

The term “portion” as utilized herein refers to a section or region of a component, without necessarily indicating any physical difference between two or more portions apart from location on the component such as “upper portion” and “lower portion”.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. The terms “steps”, “methods”, “techniques” and “functions” may be used interchangeably herein. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art after reviewing the present disclosure and are within the following claims.

What is claimed is:

1. A method for detecting dissolved inorganic carbon (DIC) in a liquid, comprising:
directing liquid through a conduit;
acidifying a portion of the liquid to a pH at least as low as 4.0 to form an acidified liquid portion;
exposing a first surface of a membrane to the acidified liquid portion, the membrane being permeable to CO₂ gas but impermeable to the liquid;
exposing a second surface of the membrane to a first headspace to collect CO₂ passed through the membrane;
quantifying the amount of CO₂ in the headspace utilizing a pressure sensor to obtain a sensed CO₂ pressure; and
determining the amount of DIC in the liquid based on the sensed CO₂ pressure.

2. The method of claim 1 wherein the pressure sensor is a differential pressure sensor positioned between the first headspace and a second headspace which lacks CO₂.

3. The method of claim 1 wherein the acidifying is accomplished by electro-acidification.

4. The method of claim 1 wherein the acidifying is accomplished by utilizing at least one reagent.

5. A system to detect dissolved inorganic carbon (DIC) in a liquid, the system disposed in a housing and comprising:
a conduit having a first opening to receive a liquid, a passage, and a second opening to discharge the liquid;
an acidification module configured to interact with the liquid in the passage to acidify a portion of the liquid to a pH at least as low as 4.0 to form an acidified liquid portion;
a membrane having a first membrane surface configured to be exposed to the acidified liquid portion, the membrane being permeable to CO₂ gas but impermeable to the liquid and having a second membrane surface configured to be exposed to a first headspace to collect CO₂ passed through the membrane;
a pressure sensor configured to quantify the amount of CO₂ in the headspace to obtain a sensed CO₂ pressure; and
a microprocessor configured to determine the amount of DIC in the liquid based on the sensed CO₂ pressure.

6. The system of claim 1 wherein the pressure sensor is a differential pressure sensor positioned between the first headspace and a second headspace which lacks CO₂.
